(19) United States
(12) Patent Application Publication
(10) Pub. No.: US 2003/0166018 A1
(43) Pub. Date: Sep. 4, 2003

(54) Methods for identifying agents that modulate mast cell degranulation

(76) Inventors: Matthias Wabl, San Francisco, CA (US); Rolf Jessberger, White Plains, NY (US)

Correspondence Address: BOZICEVIC, FIELD & FRANCIS LLP, 200 MIDDLEFIELD RD, SUITE 200, MENLO PARK, CA, 94025, US

(21) Appl. No.: 10/087,144

(22) Filed: Mar. 1, 2002

(51) Int. Cl. G01N 33/567

(52) U.S. Cl. 435/7.21

(57) ABSTRACT

The invention provides screening methods to identify agents that modulate a level or an activity of a SWAP-70 protein in a degranulation competent cell; agents that reduce a level or an activity of a SWAP-70 protein, including agents identified by the screening methods; and pharmaceutical compositions comprising such agents. The present invention further provides methods of reducing degranulation in a degranulation competent cell, such as a mast cell or basophil. The methods generally involve reducing a level or activity of a SWAP-70 protein in a degranulation competent cell. The invention further provides methods of treating a mast cell-mediated disorder in an individual.

### STATEMENT REGARDING FEDERALLY SPONSORED RESEARCH

The United States government may have certain rights in this invention pursuant to Grant No. AG20684 awarded by the National Institutes of Health.

### FIELD OF THE INVENTION

This invention is in the field of allergies and in particular mast cell degranulation.

### BACKGROUND OF THE INVENTION

Approximately 5 to 10% of the world population suffers from allergies, and in the United States, this figure may be as high as 15%. Of the approximately 40 million allergy sufferers in the United States, about 9.9 million have asthma. Asthma is the most frequent chronic condition for those under age 18. Allergic individuals synthesize IgE in response to foreign substances known as allergens. IgE antibodies are specific to the allergen to which they are elicted. The IgE antibodies bind to IgE receptors on the surface of mast cells in tissues and basophilic granulocytes in the blood. A polyvalent allergen can crosslink the surface-bound IgE, leading to degranulation of the cells. This process results in the release of pharmacologically active agents such as histamines and prostaglandins, which in turn cause the allergic symptoms in the target organ, e.g., bronchospasm in asthma or edema in a local allergic reaction. Thus, a central cell in most common allergies is the mast cell, and a central molecule is IgE.

Mast cells are specialized hematopoietic cells that are derived from progenitor stem cells in the bone marrow (for reviews see Ref.1-3). In mice mast cells leave the bone marrow into the peripheral blood as mast cell-committed precursors (Ref. 4), and enter peripheral tissues—mostly mucosal and connective tissue—and the peritoneal fluid. Immature mast cells of an advanced developmental stage, capable of exocytosis, can be found in the blood (blood mast cells; BMC) and in the bone marrow (bone marrow mast cells; BMMC) and can be cultivated in vitro in the presence of interleukin 3 (IL-3) (Ref. 5-8). Mast cells contribute important and specific functions to the immune system. The role for which mast cells are best known is that in mediating IgE-triggered allergic reactions.

Mast cells express the high-affinity receptor for IgE (FcεRI), which after binding can be stimulated by crosslinking to release a variety of biologically active mediators such as histamine, proteoglycans, proteases, serotonin. Crosslinking also induces the synthesis of prostaglandins and leukotrienes, and the synthesis and release of cytokines. The FcεRI receptor γ-chains contain immunoreceptor tyrosine-based activation motifs (ITAMs), and signaling pathways originating at the FcεRI are similar to those generated through activation of other immunoreceptors that contain ITAMs, including the B cell receptor complex (9, 10). In brief, upon FcεRI cross-linking, the src family kinase lyn phosphorylates the ITAM, to which syk kinase is recruited. Syk becomes activated, and phosphorylates a variety of cytoplasmic proteins, some of which remain to be identified (10).

Treatments for allergy are generally aimed at three possible components: 1) avoidance or reduced exposure to the allergen, which can be very difficult especially in case of children; 2) allergen immunotherapy, which only works for some allergens, and which is frequently ineffective; and 3) pharmacotherapy (medication), which is the most effective treatment. Allergy medication either should prevent the release of allergy causing chemicals such as histamine from mast cells, or stop the response of histamine on the tissues. Most currently available medications are anti-histamines. Usually in case of allergy and asthma, doctors prescribe second generation anti-histamines such as loratadine (Claritin®), fexofenadine (Allegra®), leukotriene receptor antagonists such as zafirlukast (Accolate®), and leukotriene synthesis inhibitors such as zileutron (Zyflo®). Because the airway is more sensitive to leukotrienes produced by a number of inflammatory cells, anti-leukotriene agents are usually more effective for asthmatic conditions.

Although the second generation antihistamines are as effective as the older ones (Benadryl and Chlortrimeton) they have two potential problems. First, these drugs only counteract the effect of histamine released by mast cells in the body, which are responsible for many but not all the symptoms of allergy. Therefore, anti-histamines are very effective in decreasing the itching, sneezing, and nasal secretions, but do not provide relief from nasal stuffiness and late phase allergic reactions. A number of inflammatory mediators other than histamine, such as leukotrienes and a number of vasoactive cytokines, are also released by mast cells and basophils. These inflammatory mediators remain unaffected by anti-histamines and contribute significantly to the patho-physiology of allergy and asthma. Sometimes a combination of anti-allergic and inflammatory drugs works better, but at the same time these combinations cause adverse side effects. Secondly, in more severe allergic reactions (anaphylaxis), anti-histamines do not have therapeutic effect.

In view of the aforementioned drawbacks to currently available therapies, there is an ongoing need in the field for therapeutic agents and methods to combat allergies. The present invention addresses this need.

Literature

1. Galli, S. J. and Hammel, I., Curr. Opin. Haematol. 1994. 1: 33-39; 2. Li, L. and Krilis, S. A., Allergy 1999. 54: 306-312; 3. Galli, S. J., Curr. Opin. Haematol. 2000. 7: 32-39; 4. Rodewald, H.-R., et al. Science 1996, 271: 818-822; 5. Kitamura, Y., et al. Blood 1979. 53: 1085-1088; 6. Mitsui, H., et al. Proc. Natl. Acad. Sci. U S A 1993. 90: 735-739; 7. Valent, P., et al. Blood 1992. 80: 2237-2245; 8. Sonoda, T., et al. J. Cell. Physiol. 1982. 112: 136-140; 9. Daëron, M., Ann Rev. Immunol. 1997. 15: 203-234; 10. Turner, H. and Kinet, J.-P., Nature 1999. 402 supp.: B24-B29; 11. Borggrefe, T., et al.. Journ. Biol. Chem. 1998. 273: 17025-17035; 12. Borggrefe, T., et al. Europ. J. Immunol. 1999. 29: 1812-1822; 14. Masat, L., et al. Proc. Natl. Acad. Sci. U S A . 2000. 97: 2180-2184; 15. Borggrefe, T., et al.. Europ. J. Immunol., 2001, 31: 2467-2475; 16. Yong, L. C. J., Exp. Toxic Pathol. 1997. 49: 409-424; 17. Rottem, M., et al. Blood 1992. 9: 972-980; 18. Gompert. B. D. and Tatham, P. E. R., Meth. Enzym. 1992. 219: 178-189; 19. Iemura, A. et al. Am. J. Pathol. 1994. 144: 321-328; 20. Nelson, S. Y., et al. J. Exp. Med. 1994. 179: 1777-1787; 21. Loo, D. T. and Rillema, J. R., Meth. Cell Biol. 1998. 57: 251-264; 22. van Engeland, M., et al. Cytometry 1998. 31: 1-9; 23. Demo, S. D., et al. Cytometry 1999. 36: 340-348; 24. Grawunder, U., et al. Eur. J. Biochem. 1996. 241: 931-940.

### SUMMARY OF THE INVENTION

The invention provides screening methods to identify agents that modulate a level or an activity of a SWAP-70 protein in a degranulation competent cell; agents that reduce a level or an activity of a SWAP-70 protein, including agents identified by the screening methods; and pharmaceutical compositions comprising such agents. The present invention further provides methods of reducing degranulation in a degranulation competent cell, such as a mast cell or basophil. The methods generally involve reducing a level or activity of a SWAP-70 protein in a degranulation competent cell. The invention further provides methods of treating a mast cell-mediated disorder in an individual.

### BRIEF DESCRIPTION OF THE DRAWINGS

FIG. 1 depicts the results of immunoblot analysis of SWAP-70 expression in blood-derived mast cells (MC) and splenic B cells, activated by either LPS or anti-CD40&IL-4.

FIGS. 2A and 2B depict degranulation of mast cells, measured in the hexoseaminidase assay. FIG. 2A depicts degranulation of mast cells in pools of peritoneal cells obtained from each four 6-weeks old wildtype (+/+) and SWAP-70^{ko/ko} (−/−) mice. FIG. 2B depicts degranulation of peritoneal mast cell populations purified by percol gradient centrifugation obtained from each five 6-months old wildtype (+/+) and SWAP-70^{ko/ko} (−/−) mice.

FIGS. 3A depicts stimulation of ³H-thymidine incorporation in wildtype (+/+) and SWAP-70^{ko/ko} (−/−) mast cells by IL-3 (50 μg/ml), SCF (100 ng/ml), or both. FIG. 3B depicts radioprotection of mast cells by SCF, IL-3, or both.

FIGS. 4A-F depict degranulation of wildtype (+/+) and SWAP-70^{ko/ko} (−/−) mast cells. FIG. 4A depicts degranulation of independent blood mast cell (BMC) cultures from two mice of each genotype (wildtype, +/+, and SWAP-70^{ko/ko}, −/−) after treatment with the indicated antibodies and reagents. FIG. 4B depicts kinetics of degranluation in wildtype (+/+) and SWAP-70^{ko/ko} (−/−) BMC. FIG. 4C depicts degranulation in two independent bone marrow mast cell (BMMC) cultures of each genotype (+/+or −/−) after treatment with IgE and anti-IgE. FIG. 4D depicts release of ³H-serotonin from preloaded BMMC cultures. FIG. 4E depicts release of TNF-α from BMMC cultures, stimulated either by anti-DNP IgE, or by anti-DNP IgE and DNP-BSA. FIG. 4E depicts release of IL-6 from BMMC cultures, stimulated as in FIG. 4E.

### DEFINITIONS

As used herein, the term “degranulation competent cell” includes all cells expressing on their surface FcεRI, including mast cells and basophils, and related cell types, which cells degranulate after exposure to an appropriate signal, e.g., an agent that cross-links FcεRI on the surface of the degranulation-competent cell.

As used herein, the term “disorder associated with mast cell degranulation” refers to a disorder that is caused, directly or indirectly, by mast cell degranulation. The term also refers to a disorder of which mast cell degranulation is a symptom.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines, simians, humans, felines, canines, equines, bovines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a mast cell” includes a plurality of such cells and reference to “the agent” includes reference to one or more agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The invention provides methods of identifying agents that reduce a level or an activity of SWAP-70. Such agents reduce mast cell degranulation and are therefore useful for treating disorders associated with mast cell degranulation. The invention is based on the discovery that SWAP-70, a protein that was previously shown to be expressed in activated B cells, is expressed in mast cells and is involved in mast cell development and degranulation. The inventors discovered that SWAP-70 homozygous knockout mice have fewer mature mast cells than wild type mice, and that the immature mast cells in these animals degranulate to a much lower extent than wild type immature mast cells.

The invention further provides agents identified using the methods of the invention, and pharmaceutical compositions comprising the agents. Such agents are useful in the treatment of disorders associated with mast cell degranulation, such as allergic disorders. The present invention further provides methods of reducing degranulation of a degranulation competent cell, such as a mast cell or basophil, and methods of treating a disorder associated with mast cell degranulation.

Screening Methods

The invention provides in vitro methods of identifying agents that modulate a level or an activity of a SWAP-70 protein in a mast cell. The methods generally involve contacting a SWAP-70 protein, or a cell that produces a SWAP-70 protein, with a test agent, and determining the effect, if any, on a level or an activity of the SWAP-70 protein.

In some embodiments, the method is a cell-based method and involves contacting a cell that produces SWAP-70 with a test agent, and determining the effect, if any, of the test agent on a level of SWAP-70 mRNA. In other embodiments, the methods involve contacting a cell that produces SWAP-70 with a test agent, and determining the effect, if any, of the test agent on a level of SWAP-70 polypeptide.

In other embodiments, the method is a cell-based method and involves contacting a cell that produces SWAP-70 protein with a test agent and determining the effect, if any, of the test agent on an activity of SWAP-70. In other embodiments, the methods involve contacting a mast cell that produces SWAP-70 with a test agent, and determining the effect, if any, on degranulation.

In other embodiments, the methods are cell-free methods and involve contacting a SWAP-70 protein with a test agent and determining the effect, if any, on an activity of the SWAP-70 protein. In some of these embodiments, the methods involve contacting a sample comprising SWAP-70 protein, an interacting polypeptide, and a test agent; and

determining the effect, if any, of the test agent on the interaction between the SWAP-70 and the interacting polypeptide. In some of these embodiments, the interacting polypeptide is a polypeptide other than SWAP-70. In other embodiments, the interacting polypeptide is at least a second SWAP-70 polypeptide. In other embodiments, the methods involve contacting a SWAP-70 polypeptide with a test agent; and determining the effect, if any, of the test agent on an enzymatic activity of SWAP-70. In other embodiments, the methods involve contacting SWAP-70 polypeptide with a kinase and a test agent, and determining the effect, if any, of the test agent on phosphorylation of the SWAP-70 protein.

As used herein, the term “determining” refers to both quantitative and qualitative determinations and as such, the term “determining” is used interchangeably herein with “assaying,” “measuring,” and the like.

The term “SWAP-70” refers to an approximately 585-amino acid protein that is found in the cytoplasm of mast cells. The nucleotide sequence of mouse SWAP-70 cDNA, and the amino acid sequence of the SWAP-70 protein encoded thereby, are found under GenBank Accession No. AF053974. The nucleotide sequence of human SWAP-70 cDNA, and the amino acid sequence of the SWAP-70 protein encoded thereby, are found under GenBank Accession No. AF210818.

The term “SWAP-70” encompasses murine SWAP-70, and homologs in other mammalian species, including humans. The term “SWAP-70” further encompasses fusion proteins comprising SWAP-70 and a heterologous polypeptide (“fusion partners”), where suitable fusion partners include immunological tags such as epitope tags, including, but not limited to, hemagglutinin, FLAG, and the like; proteins that provide for a detectable signal, including, but not limited to, fluorescent proteins, enzymes (e.g., β-galactosidase, luciferase, horse radish peroxidase, etc.), and the like; polypeptides that facilitate purification or isolation of the fusion protein, e.g., metal ion binding polypeptides such as 6His tags (e.g., SWAP-70/6His), GST, and the like; polypeptides that provide for subcellular localization; and polypeptides that provide for secretion from a cell. The term “SWAP-70” further encompasses polypeptides having insertions, deletions, or substitutions (including conservative substitutions) of one or more amino acids compared to a naturally-occurring SWAP-70 protein, while retaining at least one activity of naturally-occurring SWAP-70 protein.

As used herein, the term “an activity of SWAP-70” refers to any activity of a SWAP-70 protein, including but not limited to, an enzymatic activity; a protein binding activity (e.g., binding of a SWAP-70 protein to a non-SWAP-70 protein); a multimerization activity (e.g., formation of dimers, trimers, tetramers, and other multimers with one more more additional SWAP-70 molecules); a signal transduction activity; degranulation of a mast cell; and a modification of SWAP-70 that affects its activity, which modifications include, but are not limited to, phosphorylation (e.g., phosphorylation by a SYK kinase), glycosylation, and the like.

Where the assay is an in vitro cell-free assay, the methods generally involve contacting a SWAP-70 polypeptide with a test agent. The SWAP-70 polypeptide may be, but need not be, purified. For example, the SWAP-70 polypeptide can be in a cell lysate, or may be isolated, or partially purified. Thus, the assay can be conducted in the presence of additional components, as long as the additional components do not adversely affect the reaction to an unacceptable degree.

Where the assay is an in vitro cell-based assay, any of a variety of cells can be used. The cells used in the assay are usually eukaryotic cells, including, but not limited to, rodent cells, human cells, and yeast cells. The cells may be primary cell cultures or may be immortalized cell lines. The cells may be “recombinant,” e.g., the cell may have transiently or stably introduced therein a construct (e.g., a plasmid, a recombinant viral vector, or any other suitable vector) that comprises a nucleotide sequence encoding a SWAP-70 polypeptide, or that comprises a nucleotide sequence that comprises a SWAP-70 promoter operably linked to a reporter gene.

Suitable primary cell cultures include, but are not limited to, blood mast cells (BMC) and bone marrow mast cells (BMMC). In addition, primary mast cell cultures established from embryonic stem cells can be used. For example, primary mast cell cultures can be established from embryonic stem cells by cultivating the cells in IL-3 and SCF containing medium, yielding ESMC cultures (embryonic stem cell—derived mast cells; Garrington et al. (2000) EMBO J. 5387-5395). In some embodiments, mast cells or mast cell lines are used. Suitable established mast cell lines include, but are not limited to, the rat basophilic leukemia line RBL-2H3 (Barsumian et al. (1981) Eur. J. Immunol. 11: 317-323); and the P815 mastocytoma line (Moritz et al. (1998) J. Immunology 161: 4866-4874). In addition, cell lines established from mast cell tumors can be used. Other suitable cell lines include mammalian cell lines such as CHO cells, 293 cells, and the like. In some embodiments, the cell is a degranulation-competent cell, such as a mast cell or a basophil. In many embodiments, B cells or B cell lines are not used.

The terms “candidate agent,” “test agent,” “agent”, “substance” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Assays of the invention include controls, where suitable controls include a sample (e.g., a sample comprising SWAP-70 protein, or a cell that synthesizes SWAP-70) in the absence of the test agent. Generally a plurality of assay mixtures is run in parallel with different agent concentrations to obtain a differential response to the various concentrations. Typically, one of these concentrations serves as a negative control, i.e. at zero concentration or below the level of detection.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components of the assay mixture are added in any order that provides for the requisite binding or other activity. Incubations are performed at any suitable temperature, typically between 4° C. and 40° C. Incubation periods are selected for optimum activity, but may also be optimized to facilitate rapid high-throughput screening. Typically between 0.1 and 1 hour will be sufficient.

The screening methods may be designed a number of different ways, where a variety of assay configurations and protocols may be employed, as are known in the art. For example, one of the components may be bound to a solid support, and the remaining components contacted with the support bound component. The above components of the method may be combined at substantially the same time or at different times.

Where the assay is a binding assay, following the contact and incubation steps, the subject methods will generally, though not necessarily, further include a washing step to remove unbound components, where such a washing step is generally employed when required to remove label that would give rise to a background signal during detection, such as radioactive or fluorescently labeled non-specifically bound components. Following the optional washing step, the presence of bound complexes will then be detected.

A test agent of interest is one that reduces a level of SWAP-70 protein or inhibits a SWAP-70 activity by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent.

Methods of Detecting Agents that Reduce a Level of SWAP-70 mRNA and/or SWAP-70 Polypeptide

The subject screening methods include methods of detecting an agent that modulates a level of a SWAP-70 mRNA and/or SWAP-70 polypeptide in a mast cell or basophil. In some embodiments, the methods involve contacting a cell that produces SWAP-70 with a test agent, and determining the effect, if any, of the test agent on the level of SWAP-70 mRNA in the cell.

A candidate agent is assessed for any cytotoxic activity it may exhibit toward the cell used in the assay, using well-known assays, such as trypan blue dye exclusion, an MTT (3-(4,5-dimethylthiazol-2-yl)-2,5-diphenyl-2 H-tetrazolium bromide) assay, and the like. Agents that do not exhibit cytotoxic activity are considered candidate agents.

A wide variety of cell-based assays may be used for identifying agents which reduce a level of SWAP-70 mRNA in a eukaryotic cell, using, for example, a mast cell or basophil that normally produces SWAP-70 mRNA, a mammalian cell transformed with a construct comprising a SWAP-70-encoding cDNA such that the cDNA is overexpressed, or, alternatively, a construct comprising a SWAP-70 promoter operably linked to a reporter gene.

Accordingly, the present invention provides a method for identifying an agent, particularly a biologically active agent, that reduces a level of SWAP-70 expression in a cell, the method comprising: combining a candidate agent to be tested with a cell comprising a nucleic acid which encodes a SWAP-70 polypeptide, or a construct comprising a SWAP-70 promoter operably linked to a reporter gene; and determining the effect of said agent on SWAP-70 expression. A decrease of at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, in the level (i.e., an amount) of SWAP-70 mRNA and/or polypeptide following contacting the cell with a candidate agent being tested, compared to a control to which no agent is added, is an indication that the agent modulates SWAP-70 expression.

SWAP-70 mRNA and/or polypeptide whose levels are being measured can be encoded by an endogenous SWAP-70 polynucleotide, or the SWAP-70 polynucleotide can be one that is comprised within a recombinant vector and introduced into the cell, i.e., the SWAP-70 mRNA and/or polypeptide can be encoded by an exogenous SWAP-70 polynucleotide. For example, a recombinant vector may comprise an isolated SWAP-70 transcriptional regulatory sequence, such as a promoter sequence, operably linked to a reporter gene (e.g,. β-galactosidase, chloramphenicol acetyl transferase, a fluorescent protein, luciferase, or other gene that can be easily assayed for expression).

In these embodiments, the method for identifying an agent that modulates a level of SWAP-70 expression in a cell, comprises: combining a candidate agent to be tested with a cell comprising a nucleic acid which comprises a SWAP-70 gene transcriptional regulatory element operably linked to a reporter gene; and determining the effect of said agent on reporter gene expression. A recombinant vector may comprise an isolated SWAP-70 transcriptional regulatory sequence, such as a promoter sequence, operably linked to sequences coding for a SWAP-70 polypeptide; or the transcriptional control sequences can be operably linked to coding sequences for a SWAP-70 fusion protein comprising SWAP-70 polypeptide fused to a polypeptide which facilitates detection. In these embodiments, the method comprises combining a candidate agent to be tested with a cell comprising a nucleic acid which comprises a SWAP-70 gene transcriptional regulatory element operably linked to a SWAP-70 polypeptide-coding sequence; and determining the effect of said agent on SWAP-70 expression, which determination can be carried out by measuring an amount of SWAP-70 mRNA, SWAP-70 polypeptide, or SWAP-70 fusion polypeptide produced by the cell.

Cell-based assays generally comprise the steps of contacting the cell with an agent to be tested, forming a test sample, and, after a suitable time, assessing the effect of the agent on SWAP-70 expression. A control sample comprises the same cell without the candidate agent added. SWAP-70 expression levels are measured in both the test sample and the control sample. A comparison is made between SWAP-70 expression level in the test sample and the control sample. SWAP-70 expression can be assessed using conventional assays. For example, when a mammalian cell line is transformed with a construct that results in expression of SWAP-70, SWAP-70 mRNA levels can be detected and measured, or SWAP-70 polypeptide levels can be detected and measured. A suitable period of time for contacting the agent with the cell can be determined empirically, and is generally a time sufficient to allow entry of the agent into the cell and to allow the agent to have a measurable effect on SWAP-70 mRNA and/or polypeptide levels. Generally, a suitable time is between 10 minutes and 24 hours, or from about 1 hour to about 8 hours.

Methods of measuring SWAP-70 mRNA levels are known in the art, several of which have been described above, and any of these methods can be used in the methods of the present invention to identify an agent which modulates SWAP-70 mRNA level in a cell, including, but not limited to, a PCR, such as a PCR employing detectably labeled oligonucleotide primers, and any of a variety of hybridization assays.

Similarly, SWAP-70 polypeptide levels can be measured using any standard method, several of which have been described herein, including, but not limited to, an immunoassay such as ELISA, for example an ELISA employing a detectably labeled antibody specific for a SWAP-70 polypeptide.

SWAP-70 polypeptide levels can also be measured in cells harboring a recombinant construct comprising a nucleotide sequence that encodes a SWAP-70 fusion protein, where the fusion partner provides for a detectable signal or can otherwise be detected. For example, where the fusion partner provides an immunologically recognizable epitope (an “epitope tag”), an antibody specific for an epitope of the fusion partner can be used to detect and quantitate the level of SWAP-70. In some embodiments, the fusion partner provides for a detectable signal, and in these embodiments, the detection method is chosen based on the type of signal generated by the fusion partner. For example, where the fusion partner is a fluorescent protein, fluorescence is measured. Fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; and the like. Where the fusion partner is an enzyme that yields a detectable product, the product can be detected using an appropriate means, e.g., β-galactosidase can, depending on the substrate, yield colored product, which is detected spectrophotometrically, or a fluorescent product; luciferase can yield a luminescent product detectable with a luminometer; etc.

A number of methods are available for analyzing nucleic acids for the presence and/or level of a specific mRNA in a cell. The mRNA may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki, et al. (1985), Science 239:487, and a review of techniques maybe found in Sambrook, et al. Molecular Cloning: A Laboratory Manual, CSH Press 1989, pp.14.2-14.33. Alternatively, various methods are known in the art that utilize oligonucleotide ligation as a means of detecting polymorphisms, for examples see Riley et al. (1990), Nucl. Acids Res. 18:2887-2890; and Delahunty et al. (1996), Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′, 7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

A variety of different methods for determining the nucleic acid abundance in a sample are known to those of skill in the art, where particular methods of interest include those described in: Pietu et al., Genome Res. (June 1996) 6: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

A number of methods are available for determining the expression level of a gene or protein in a particular sample. For example, detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. Alternatively, the secondary antibody conjugated to a fluorescent compound, e.g. fluorescein, rhodamine, Texas red, etc. The absence or presence of antibody binding may be determined by various methods, including flow cytometry of dissociated cells, microscopy, radiography, scintillation counting, etc.

Methods of Detecting Agents that Inhibit SWAP-70 Binding to other Proteins

In some embodiments, the invention provides methods of identifying an agent that inhibits the interaction of a SWAP-70 polypeptide with another polypeptide. The methods generally involve contacting a SWAP-70 polypeptide and an interacting polypeptide with a test agent, and determining the effect, if any, of the test agent on the interaction of SWAP-70 with the interacting polypeptide. In some embodiments, the interacting protein is a protein other than a SWAP-70 protein. In other embodiments, the interacting protein is at least a second SWAP-70 protein. In some embodiments, methods of identifying an agent that inhibits the interaction of a SWAP-70 polypeptide with an interacting polypeptide are cell-based methods. In other embodiments, methods of identifying an agent that inhibits the interaction of a SWAP-70 polypeptide with an interacting polypeptide are cell-free methods.

Formation of a binding complex between SWAP-70 and an interacting polypeptide can be detected using any known method. Suitable methods include: a yeast two-hybrid method; a FRET assay; a BRET assay; a fluorescence quenching assay; a fluorescence anisotropy assay; an immunological assay; and an assay involving binding of a detectably labeled protein to an immobilized protein.

Immunological assays, and assays involving binding of a detectably labeled protein to an immobilized protein can be arranged in a variety of ways. Immunoprecipitation assays can be designed, wherein the SWAP-70/interacting polypeptide complex is detected by precipitating the complex with antibody specific for SWAP-70 and/or the interacting polypeptide.

In other embodiments, the assay is a binding assay which detects binding of SWAP-70 to an immobilized protein, or which detects binding of a protein to immobilized SWAP-70 protein. In some embodiments, the SWAP-70 polypeptide is labeled with a detectable label, and binding to an immobilized interacting polypeptide is detected. In other embodiments, the interacting polypeptide is labeled with a detectable label, and binding to an immobilized SWAP-70 polypeptide is detected. In other embodiments, the SWAP-70 polypeptide is immobilized, and binding of the interacting polypeptide to the SWAP-70 polypeptide is detected using an antibody specific for the interacting polypeptide, where the antibody is either directly labeled or a secondary antibody that is labeled is used. In other embodiments, the interacting polypeptide is immobilized, and binding of the SWAP-70 polypeptide to the interacting polypeptide is detected using an antibody specific for the SWAP-70 polypeptide, where the antibody is either directly labeled or a secondary antibody that is labeled is used.

Formation of a binding complex between a SWAP-70 polypeptide and a SWAP-70-interacting polypeptide can also be detected using fluorescence resonance energy transfer (FRET), bioluminescence resonance energy transfer (BRET), anisotropy measurements, and fluorescence quenching measurements.

FRET involves the transfer of energy from a donor fluorophore in an excited state to a nearby acceptor fluorophore. For this transfer to take place, the donor and acceptor molecules must in close proximity (e.g., less than 10 nanometers apart, usually between 10 and 100 Å apart), and the emission spectra of the donor fluorophore must overlap the excitation spectra of the acceptor fluorophore. In these embodiments, a fluorescently labeled SWAP-70 protein serves as a donor and/or acceptor in combination with a second fluorescent protein or dye, e.g., a fluorescent protein as described in Matz et al., Nature Biotechnology (October 1999) 17:969-973; a green fluorescent protein (GFP), including a “humanized” GFP; a GFP from Aequoria victoria or fluorescent mutant thereof, e.g., as described in U.S. Pat. Nos. 6,066,476; 6,020,192; 5,985,577; 5,976,796; 5,968,750; 5,968,738; 5,958,713; 5,919,445; 5,874,304, the disclosures of which are herein incorporated by reference; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); other fluorescent dyes, e.g., coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye, etc., chemilumescent dyes, e.g., luciferases.

BRET is a protein-protein interaction assay based on energy transfer from a bioluminescent donor to a fluorescent acceptor protein. The BRET signal is measured by the amount of light emitted by the acceptor to the amount of light emitted by the donor. The ratio of these two values increases as the two proteins are brought into proximity. The BRET assay has been amply described in the literature. See, e.g., U.S. Pat. Nos. 6,020,192; 5,968,750; and 5,874,304; and Xu et al. (1999) Proc. Natl. Acad. Sci. USA 96:151-156. BRET assays maybe performed by analyzing transfer between a bioluminescent donor protein and a fluorescent acceptor protein. Interaction between the donor and acceptor proteins can be monitored by a change in the ratio of light emitted by the bioluminescent and fluorescent proteins. In this application, the SWAP-70 protein serves as donor and/or acceptor protein.

Fluorescent SWAP-70 can be produced by generating a construct comprising a SWAP-70 and a fluorescent fusion partner, e.g., a fluorescent protein as described in Matz et al. ((1999) Nature Biotechnology 17:969-973), a green fluorescent protein from any species or a derivative thereof; e.g., a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); a GFP from Aequoria victoria or fluorescent mutant thereof, e.g., as described in U.S. Pat. Nos. 6,066,476; 6,020,192; 5,985,577; 5,976,796; 5,968,750; 5,968,738; 5,958,713; 5,919,445; 5,874,304. Generation of such a construct, and production of a SWAP-70/fluorescent protein fusion protein is well within the skill level of those of ordinary skill in the art.

Alternatively, binding may be assayed by fluorescence anisotropy. Fluorescence anisotropy assays are amply described in the literature. See, e.g., Jameson and Sawyer (1995) Methods Enzymol. 246:283-300.

Fluorescent SWAP-70 protein can also be generated by producing SWAP-70 in an auxotrophic strain of bacteria which requires addition of one or more amino acids in the medium for growth. A SWAP-70-encoding construct that provides for expression in bacterial cells is introduced into the auxotrophic strain, and the bacteria are cultured in the presence of a fluorescent amino acid, which is incorporated into the SWAP-70 protein produced by the bacterium. SWAP-70 is then purified from the bacterial culture using standard methods for protein purification.

Where the interacting protein is at least a second SWAP-70 protein, the effect of the test agent on binding can be determined by determining the effect on multimerization of SWAP-70. As used herein, the term “multimerization” refers to formation of dimers, trimers, tetramers, and higher multimers of SWAP-70. Whether a SWAP-70 forms a complex with one or more additional SWAP-70 molecules can be determined using any known assay, including assays as described above for interacting proteins. Formation of multimers can also be detected using non-denaturing gel electrophoresis, where multimerized SWAP-70 migrates more slowly than monomeric SWAP-70. Formation of multimers can also be detected using fluorescence quenching techniques.

Formation of multimers can also be detected by analytical ultracentrifugation, for example through glycerol or sucrose gradients, and subsequent visualization of SWAP-70 in gradient fractions by Western blotting or staining of SDS-polyacrylamide gels. Multimers are expected to sediment at defined positions in such gradients. Formation of multimers can also be detected using analytical gel filtration, e.g. in HPLC or FPLC systems, e.g. on columns such as Superdex 200 (Pharmacia Amersham Inc.). Multimers run at defined positions on these columns, and fractions can be analyzed as above. The columns are highly reproducible, allowing one to relate the number and position of peaks directly to the multimerization status of the protein.

Identification of Proteins that Bind SWAP-70 Protein

The present invention further provides methods of identifying or screening for proteins which interact with SWAP-70, or derivatives, fragments or analogs thereof. In specific embodiments, the method of identifying a molecule that binds to a SWAP-70 comprises contacting the SWAP-70 with a plurality of molecules under conditions conducive to binding between the SWAP-70 and the molecules; and identifying a molecule within the plurality that binds to the SWAP-70. The SWAP-70 in the method can either be a purified or non-purified form. A protein that interacts with a SWAP-70 protein can be identified using a FRET assay, a BRET assay, a yeast two-hybrid assay, and the like. BRET and FRET assays are described above. Many of these techniques are amenable to high through-put screens.

In some embodiments, the method of identifying or screening is a yeast two-hybrid assay system or a variation thereof, as further described below. The yeast two-hybrid screen has been described in the literature. See, e.g., Zhu and Kahn (1997) Proc. Natl. Acad. Sci. U.S.A. 94:13063-13068; Fields and Song (1989) Nature 340:245-246; and U.S. Pat. No. 5,283,173; Chien et al. (1991) Proc. Natl. Acad. Sci. U.S.A. 88:9578-9581.

Derivatives (e.g., fragments) and analogs of a SWAP-70 can also be assayed for binding to a binding partner by any method known in the art, for example, immunoprecipitation with an antibody that binds to the protein in a complex followed by analysis by size fractionation of the immunoprecipitated proteins (e.g. by denaturing or nondenaturing polyacrylamide gel electrophoresis), Western analysis, non-denaturing gel electrophoresis, etc.

One aspect of the present invention provides methods for assaying and screening fragments, derivatives and analogs of SWAP-70 for interacting proteins (for binding to a SWAP-70 polypeptide). Derivatives, analogs and fragments of proteins that interact with a SWAP-70 are readily identified by means of a yeast two-hybrid assay system. Because the interactions are screened for in yeast, the intermolecular protein interactions detected in this system occur under physiological conditions that mimic the conditions in eukaryotic cells. This feature facilitates identification of proteins capable of interaction with a SWAP-70 polypeptide from a variety of mammalian species. Identification of interacting proteins by a yeast two-hybrid system is based upon the detection of expression of a reporter gene, the transcription of which is dependent upon the reconstitution of a transcriptional regulator by the interaction of two proteins, each fused to one half of the transcriptional regulator. The “bait” (i.e., SWAP-70 polypeptide or derivative or analog thereof) and “prey” (proteins to be tested for ability to interact with the bait) proteins are expressed as fusion proteins to a DNA binding domain, and to a transcriptional regulatory domain, respectively, or vice versa.

In various specific embodiments, the prey has a complexity of at least about 50, about 100, about 500, about 1,000, about 5,000, about 10,000, or about 50,000; or has a complexity in the range of about 25 to about 100,000, about 100 to about 100,000, about 50,000 to about 10⁵, about 10⁵ to about 5×10⁵, about 10⁶ to about 10⁷, or about 10⁷ to about 5×10⁷, or more. For example, the prey population can be one or more nucleic acids encoding mutants of a protein (e.g., as generated by site-directed mutagenesis or another method of making mutations in a nucleotide sequence).

In some embodiments, the prey populations are proteins encoded by DNA, e.g., cDNA or genomic DNA or synthetically-generated DNA. For example, the populations can be expressed from chimeric genes comprising cDNA sequences from an un-characterized sample of a population of cDNA from mRNA. One characteristic of the yeast two-hybrid system is that proteins examined in this system are expressed as cytoplasmic proteins, and therefore do not pass through the secretory pathway.

In a specific embodiment, recombinant biological libraries expressing random peptides can be used as the source of prey nucleic acids. In another embodiment, the invention provides methods of screening for inhibitors or enhancers of the protein interactants identified herein. Briefly, the protein-protein interaction assay can be carried out as described herein, except that it is done in the presence of one or more candidate molecules. An increase or decrease in reporter gene activity relative to that present when the one or more candidate molecules are absent indicates that the candidate molecule has an effect on the interacting pair. In one embodiment, inhibition of the interaction is selected for (i.e., inhibition of the interaction is necessary for the cells to survive), for example, where the interaction activates the URA3 gene, causing yeast to die in medium containing the chemical 5-fluoroorotic acid (Rothstein, 1983, Meth. Enzymol. 101:167-180).

The identification of inhibitors of such interactions can also be accomplished, for example, but not by way of limitation, using competitive inhibitor assays, as described above. In general, proteins of the bait and prey populations are provided as fusion (chimeric) proteins (e.g., by recombinant expression of a chimeric coding sequence) comprising each protein contiguous to a pre-selected sequence. For one population, the pre-selected sequence is a DNA binding domain. The DNA binding domain can be any DNA binding domain, as long as it specifically recognizes a DNA sequence within a promoter. For example, the DNA binding domain is of a transcriptional activator or inhibitor. For the other population, the pre-selected sequence is an activator or inhibitor domain of a transcriptional activator or inhibitor, respectively.

The regulatory domain alone (not as a fusion to a protein sequence) and the DNA-binding domain alone (not as a fusion to a protein sequence) preferably do not detectably interact (so as to avoid false positives in the assay). The assay system further includes a reporter gene operably linked to a promoter that contains a binding site for the DNA binding domain of the transcriptional activator (or inhibitor). Accordingly, in the present method of the invention, binding of a SWAP-70 polypeptide to a prey fusion protein leads to reconstitution of a transcriptional activator (or inhibitor) which activates (or inhibits) expression of the reporter gene. The activation (or inhibition) of transcription of the reporter gene occurs intracellularly, e.g., in prokaryotic or eukaryotic cells, preferably in cell culture.

The promoter that is operably linked to the reporter gene nucleotide sequence can be a native or non-native promoter of the nucleotide sequence, and the DNA binding site(s) that are recognized by the DNA binding domain portion of the fusion protein can be native to the promoter (if the promoter normally contains such binding site(s)) or non-native to the promoter. Thus, for example, one or more tandem copies (e.g., four or five copies) of the appropriate DNA binding site can be introduced upstream of the TATA box in the desired promoter (e.g., in the area of about position −100 to about −400). In one embodiment, 4 or 5 tandem copies of the 17 bp UAS (GAL4 DNA binding site) are introduced upstream of the TATA box in the desired promoter, which is upstream of the desired coding sequence for a selectable or detectable marker.

In one embodiment, the GAL1-10 promoter is operably fused to the desired nucleotide sequence; the GAL1-10 promoter already contains 4 binding sites for GAL4. Alternatively, the transcriptional activation binding site of the desired gene(s) can be deleted and replaced with GAL4 binding sites (Bartel et al., 1993, BioTechniques 14:920-924; Chasman et al., 1989, Mol. Cell. Biol. 9:4746-4749). The reporter gene may contain the sequence encoding a detectable or selectable marker, the expression of which is regulated by the transcriptional activator, such that the marker is either turned on or off in the cell in response to the presence of a specific interaction. In one embodiment, the assay is carried out in the absence of background levels of the transcriptional activator (e.g., in a cell that is mutant or otherwise lacking in the transcriptional activator).

In one embodiment, more than one reporter gene is used to detect transcriptional activation, e.g., one reporter gene encoding a detectable marker and one or more reporter genes encoding different selectable markers. The detectable marker can be any molecule that can give rise to a detectable signal, e.g., a fluorescent protein or a protein that can be readily visualized or that is recognizable by a specific antibody. The selectable marker can be any protein molecule that confers the ability to grow under conditions that do not support the growth of cells not expressing the selectable marker, e.g., the selectable marker is an enzyme that provides an essential nutrient and the cell in which the interaction assay occurs is deficient in the enzyme and the selection medium lacks such nutrient. The reporter gene can either be under the control of the native promoter that naturally contains a binding site for the DNA binding protein, or under the control of a heterologous or synthetic promoter.

The activation domain and DNA binding domain used in the assay can be from a wide variety of transcriptional activator proteins, as long as these transcriptional activators have separable binding and transcriptional activation domains. For example, the GAL4 protein of S. cerevisiae (Ma et al., 1987, Cell 48:847-853), the GCN4 protein of S. cerevisiae (Hope and Struhl, 1986, Cell 46:885-894), the ARD1 protein of S. cerevisiae (Thukral et al., 1989, Mol. Cell. Biol. 9:2360-2369), and the human estrogen receptor (Kumar et al., 1987, Cell 51:941-951), have separable DNA binding and activation domains. The DNA binding domain and activation domain that are employed in the fusion proteins need not be from the same transcriptional activator. In a specific embodiment, a GAL4 or LEXA DNA binding domain is employed. In another specific embodiment, a GAL4 or herpes simplex virus VP16 (Triezenberg et al., 1988, Genes Dev. 2:730-742) activation domain is employed.

To facilitate isolation of the encoded proteins, the fusion constructs can further contain sequences encoding affinity tags such as glutathione-S-transferase or maltose-binding protein or an epitope of an available antibody, for affinity purification (e.g., binding to glutathione, maltose, or a particular antibody specific for the epitope, respectively) (Allen et al., 1995, TIBS 20:511-516). In another embodiment, the fusion constructs further comprise bacterial promoter sequences for recombinant production of the fusion protein in bacterial cells.

The host cell in which the interaction assay occurs can be any cell, prokaryotic or eukaryotic, in which transcription of the reporter gene can occur and be detected, including, but not limited to, mammalian (e.g., monkey, mouse, rat, human, bovine), chicken, bacterial, or insect cells, and is generally a yeast cell. Expression constructs encoding and capable of expressing the binding domain fusion proteins, the transcriptional activation domain fusion proteins, and the reporter gene product(s) are provided within the host cell, by mating of cells containing the expression constructs, or by cell fusion, transformation, electroporation, microinjection, etc.

In a specific embodiment in which the assay is carried out in mammalian cells (e.g., hamster cells, HeLa cells), the DNA binding domain is the GAL4 DNA binding domain, the activation domain is the herpes simplex virus VP16 transcriptional activation domain, and the reporter gene contains the desired coding sequence operably linked to a minimal promoter element from the adenovirus E1B gene driven by several GAL4 DNA binding sites (see Fearon et al., 1992, Proc. Natl. Acad. Sci. U.S.A. 89:7958-7962). The host cell used should not express an endogenous transcription factor that binds to the same DNA site as that recognized by the DNA binding domain fusion population.

In some embodiments, the host cell is mutant or otherwise lacking in an endogenous, functional form of the reporter gene(s) used in the assay. Various vectors and host strains for expression of the two fusion protein populations in yeast are known and can be used (see e.g., U.S. Pat. No. 5,1468,614; Bartel et al., 1993, “Using the two-hybrid system to detect protein-protein interactions” In Cellular Interactions in Development, Hartley, ed., Practical Approach Series xviii, IRL Press at Oxford University Press, New York, N.Y., pp. 153-179; Fields and Stemglanz, 1994, Trends In Genetics 10:286-292).

By way of example but not limitation, yeast strains or derivative strains made therefrom, which can be used are N105, N106, N1051, N1061, and YULH. Other exemplary strains that can be used in the assay of the invention also include, but are not limited to, the following: Y190: MATa, ura3-52, his3-200, lys2-801, ade2-101, trp1-901, leu2-3,112, gal4α, gal80α, cyhr 2, LYS2:: GAL1UAS -HIS3TATA HIS3, URA3:: GAL1UAS -GAL1TATA -lacZ; Harper et al., 1993, Cell 75:805-816, available from Clontech, Palo Alto, Calif. Y190 contains HIS3 and lacZ reporter genes driven by GAL4 binding sites. CG-1945: MATa, ura3-52, his3-200, lys2-801, ade2-101, trp1-901, leu2-3,112, gal4-542, gal80-538, cyhr2, LYS2:: GAL1UAS -HIS3TATA HIS3, URA3:: GAL1UAS17mers(×3) -CYC1TATA -lacZ, available from Clontech, Palo Alto, Calif. CG-1945 contains HIS3 and lacZ reporter genes driven by GAL4 binding sites. Y187: MAT-α, ura3-52, his3-200, ade2-101, trp1-901, leu2-3,112, gal4α, gal80α, URA3:: GAL1UAS -GAL1TATA -lacZ, available from Clontech, Palo Alto, Calif. Y187 contains a lacZ reporter gene driven by GAL4 binding sites. SFY526: MATa, ura3-52, his3-200, lys2-801, ade2-101, trp1-901, leu2-3,112, gal4-542, gal80-538, can^{r}, URA3:: GAL1-lacZ, available from Clontech, Palo Alto, Calif. SFY526 contains HIS3 and lacZ reporter genes driven by GAL4 binding sites. HF7c: MATa, ura3-52, his3-200, lys2-801, ade2-101, trp1-901, leu2-3,112, gal4-542, gal80-538, LYS2:: GAL1-HIS3, URA3:: GAL1UAS17MERS(×3) -CYC1-lacZ, available from Clontech, Palo Alto, Calif. HF7c contains HIS3 and lacZ reporter genes driven by GAL4 binding sites. YRG-2: MATa, ura3-52, his3-200, lys2-801, ade2-101, trp1-901, leu2-3,112, gal4-542, gal80-538, LYS2:: GAL1UAS -GAL1TATA -HIS3, URA3:: GAL1UAS17mers(×3) -CYC1-lacZ, available from Stratagene, La Jolla, Calif. YRG-2 contains HIS3 and lacZ reporter genes driven by GAL4 binding sites.

Many other strains commonly known and available in the art can be used. If not already lacking in endogenous reporter gene activity, cells mutant in the reporter gene may be selected by known methods, or the cells can be made mutant in the target reporter gene by known gene-disruption methods prior to introducing the reporter gene (Rothstein, 1983, Meth. Enzymol. 101:202-211).

In a specific embodiment, plasmids encoding the different fusion protein populations can be introduced simultaneously into a single host cell (e.g., a haploid yeast cell) containing one or more reporter genes, by co-transformation, to conduct the assay for protein-protein interactions. Alternatively, the two fusion protein populations are introduced into a single cell either by mating (e.g., for yeast cells) or cell fusions (e.g. of mammalian cells). In a mating type assay, conjugation of haploid yeast cells of opposite mating type that have been transformed with a binding domain fusion expression construct (usually a plasmid) and an activation (or inhibitor) domain fusion expression construct (usually a plasmid), respectively, will deliver both constructs into the same diploid cell.

In a specific embodiment, transcription of the reporter gene is detected by a linked replication assay. For example, as described by Vasavada et al., 1991, Proc. Natl. Acad. Sci. U.S.A. 88:10686-10690, expression of SV40 large T antigen is under the control of the ElB promoter responsive to GAL4 binding sites. The replication of a plasmid containing the SV40 origin of replication, indicates a protein-protein interaction. Alternatively, a polyoma virus replicon can be used (Vasavada et al., 1991, Proc. Natl. Acad. Sci. U.S.A. 88:10686-90).

In another embodiment, the expression of reporter genes that encode proteins can be detected by immunoassay, i.e., by detecting the immunospecific binding of an antibody to such protein, which antibody can be labeled, or incubated with a labeled binding partner to the antibody, to yield a detectable signal. Alam and Cook disclose non-limiting examples of detectable marker genes that can be operably linked to a transcriptional regulatory region responsive to a reconstituted transcriptional activator, and thus used as reporter genes (Alam and Cook, 1990, Anal. Biochem. 188:245-254).

The activation of reporter genes like URA3 or HIS3 enables the cells to grow in the absence of uracil or histidine, respectively, and hence serves as a selectable marker. Thus, after mating, the cells exhibiting protein-protein interactions are selected by the ability to grow in media lacking a nutritional component, such as uracil or histidine (see Le Douarin et al., 1995, Nucl. Acids Res. 23:876-878; Durfee et al., 1993, Genes Dev. 7:555-569; Pierrat et al., 1992, Gene 119:237-245; Wolcott et al., 1966, Biochem. Biophys. Acta 122:532-534).

In other embodiments of the present invention, the activities of the reporter genes such as GFP or lacZ are monitored by measuring a detectable signal (e.g., fluorescent or chromogenic, respectively) that results from the activation of these reporter genes. LacZ transcription, for example, can be monitored by incubation in the presence of a substrate, such as X-gal (5-bromo-4-chloro-3-indolyl-β-D-galactoside), of its encoded enzyme, β-galactosidase. The pool of all interacting proteins isolated by this manner from mating the SWAP-70 polypeptide product and the library identifies the “SWAP-70 interactive population”. In one embodiment, false positives arising from transcriptional activation by the DNA binding domain fusion proteins in the absence of a transcriptional activator domain fusion protein are prevented or reduced by negative selection prior to exposure to the activation domain fusion population (see e.g. PCT International Publication No. WO97/47763, published Dec. 18, 1997).

Negative selection can be carried out on the recovered protein-protein complex by known methods (see e.g., Bartel et al., 1993, BioTechniques 14:920-924; PCT International Publication No. WO97/47763, published Dec. 18, 1997). In one embodiment of the invention the DNA sequences encoding the pairs of interactive proteins are isolated by a method wherein either the DNA-binding domain hybrids or the activation domain hybrids are amplified, in separate respective reactions. Generally, the amplification is carried out by polymerase chain reaction (PCR) (see U.S. Pat. Nos. 4,683,202; 4,683,195; and 4,889,818; Gyllenstein et al., 1988, Proc. Natl. Acad. Sci. U.S.A. 85:7652-7656; Ochman et al., 1988, Genetics 120:621-623; Loh et al., 1989, Science 243:217-220; Innis et al., 1990, PCR Protocols, Academic Press, Inc., San Diego, Calif.) using pairs of oligonucleotide primers specific for either the DNA-binding domain hybrids or the activation domain hybrids.

Other amplification methods known in the art can be used, including but not limited to ligase chain reaction (see EP 320,308), use of Qβ replicase, or methods listed in Kricka et al., 1995, Molecular Probing, Blotting, and Sequencing, Academic Press, New York, Chapter 1 and Table IX. The plasmids encoding the DNA-binding domain hybrid and the activation domain hybrid proteins can also be isolated and cloned by any of the methods well known in the art. For example, but not by way of limitation, if a shuttle (yeast to E. coli) vector is used to express the fusion proteins, the genes can be recovered by transforming the yeast DNA into E. coli and recovering the plasmids from E. coli (see e.g., Hoffman et al., 1987, Gene 57:267-272). Alternatively, the yeast vector can be isolated, and the insert encoding the fusion protein subcloned into a bacterial expression vector, for growth of the plasmid in E. coli.

Methods of Identifying Agents that Inhibit an Activity of SWAP-70

In some embodiments, the invention provides methods of identifying an agent that inhibits an activity of SWAP-70, including, but not limited to, a protein binding activity, an enzymatic activity, a signal transduction activity, a phosphorylation state of SWAP-70, mast cell degranulation, and the like. The methods generally comprise contacting a cell that produces SWAP-70, or a SWAP-70 polypeptide, with a test agent, and determining the effect, if any, on the activity of the SWAP-70.

In some embodiments, the activity of SWAP-70 is a protein binding activity. Assays for detecting agents that inhibit SWAP-70 binding to a non-SWAP-70 protein are described above.

In other embodiments, the activity of SWAP-70 is phosphorylation by a kinase. In these embodiments, the activity of SWAP-70 is affected by a post-translation modification, such as phosphorylation. Thus, in some embodiments, the invention provides methods of identifying an agent that inhibits phosphorylation of SWAP-70 by a kinase. The methods generally involve contacting a SWAP-70 protein with a test agent, and determining the phosphorylation state of a SWAP-70 polypeptide and/or the degree of phosphorylation of a population of SWAP-70 polypeptides. The assay can be a whole-cell assay or a cell-free assay. Where the assay is a cell-based assay, the test agent is contacted with a cell expressing SWAP-70, and the level of phosphorylation of SWAP-70 in the cells is determined. Where the assay is a cell-free assay, a test agent is contacted with SWAP-70, in the presence of a kinase that phosphorylates SWAP-70 (e.g., a SYK kinase), and the effect, if any, on the phosphorylation of SWAP-70 by the kinase is determined.

The phosphorylation state of SWAP-70 can be determined using any known assay. For example, in some embodiments, an antibody specific for a phosphorylated amino acid is used to detect phosphorylation of SWAP-70. The antibody can detect phosphorylated SWAP-70 in a variety of assay formats, including, but not limited to, immunoprecipitation, Western blotting, enzyme-linked immunosorbent assays (ELISA), and the like. In other embodiments, radioactive phosphate is used, and the level of phosphorylation of SWAP-70 is determined by measuring the amount of radioactivity of a SWAP-70 protein.

SWAP-70 phosphorylated with radioactive phosphate, by action of a kinase such as a SYK kinase, can be detected in various ways. For example, the amount of radioactivity (i.e., degree of phosphorylation) of the phosphorylated SWAP-70 can be measured using a scintillation counter. As one non-limiting example, after action of the kinase on the SWAP-70 in the presence of radioactive phosphate, the phosphorylated SWAP-70 is precipitated with an antibody specific for SWAP-70, or is isolated using some other standard method (e.g., where the SWAP-70 is a histidine tagged SWAP-70 fusion protein, e.g., SWAP-70/6His, the fusion protein is bound to an insoluble support, such as a bead, that has nickel bound, directly or via a linker, to the solid support). The radioactivity of the SWAP-70-containing immunoprecipitate or the Ni-bead-bound SWAP-70 is then counted in a scintillation counter. SWAP-70 that is phosphorylated with radioactive phosphate can also be visualized, e.g., by autoradiography following gel electrophoresis.

In some embodiments, the methods involve contacting a mast cell or other cell that is degranulation competent with a test agent, and determining the effect, if any, of the test agent on degranulation. Degranulation generally occurs in the presence of an antibody or other agent that cross-links FcεRI on the surface of the degranulation-competent cell such that the cell, in the absence of a test agent, degranulates. Accordingly, the method generally involves contacting the degranulation-competent cell with an agent that cross-links FcεRI on the surface of the degranulation-competent cell, and a test agent, and determining the effect, if any, of the test agent on degranulation.

Degranulation can be assayed using any known method, including, but not limited to, a hexosaminidase assay; surface annexin V measurement; an assay (e.g., an ELISA) to detect release of cytokines such as IL-6 and TNF-α; an assay to detect histamine release; and an assay to detect release of sphingolipids such as LTC4. Such assays are well known in the art. Several such assays are described in the Examples. An agent that cross-links FcεRI on the surface of the degranulation-competent cell such that the cell degranulates includes, but it not limited to, a combination of IgE and an antibody specific for IgE; a combination of IgE and an antigen that is bound by the IgE (e.g., anti-dinitrophenyl (DNP) IgE crosslinked with DNP coupled to bovine serum albumin (BSA)).

Mast Cells Deficient in SWAP-70

The invention further provides isolated mast cells that are deficient in SWAP-70. In many embodiments, the mast cells comprise a non-functional SWAP-70 allele. In some of these embodiments, the mast cells are heterozygous for the non-functional SWAP-70 allele. In other embodiments, the mast cells are homozygous for a non-functional SWAP-70 allele, and are referred to herein as “homozygous SWAP-70 knockout mast cells.”

Heterologous and homozygous SWAP-70 knockout mast cells are obtained using well-known methods. See, e.g., Transgenesis Techniques: Principles and Protocols D. Murphy and D. A. Carter, ed. (June 1993) Humana Press; Transgenic Animal Technology: A Laboratory Handbook C. A. Pinkert, ed. (January 1994) Academic Press; Transgenic Animals F. Grosveld and G Kollias, eds. (July 1992) Academic Press; and Embryonal Stem Cells: Introducing Planned Changes into the Animal Germline M. L. Hooper (January 1993) Gordon & Breach Science Pub. The Examples provide a description of how to generate SWAP-70 knockout mast cells.

Essentially, a construct that comprises a region of homology to an endogenous SWAP-70-encoding gene is used, such that homologous recombination between the construct and the endogenous SWAP-70 occurs. The construct comprises a mutation, e.g., a deletion, substitution, inversion, etc., compared to the endogenous SWAP-70 gene such that, upon homologous recombination with the endogenous SWAP-70 gene to form a recombinant allele, the recombinant allele is non-functional, i.e., is a knockout allele. As described in the Examples, in the knockout allele, the first exon and part of the 5′ UTR is removed. Any mutation that results in a non-functional SWAP-70 allele can be used.

As described in the Examples, immature homozygous SWAP-70 knockout mast cells retain ability to degranulate when contacted with an anti-IgE antibody that cross-link the FcεRI on the surface of the cells, albeit at a reduced level. Such cells may retain a low level ability to degranulate due to a compensatory mechanism.

Immature homozygous SWAP-70 knockout mast cells of the invention are useful for testing agents for their ability to inhibit the residual, SWAP-70 independent, degranulation in these cells. Accordingly, the invention further provides methods of identifying an agent that inhibits degranulation of a subject SWAP-70^{-/-} immature mast cell. The methods generally involve contacting the SWAP-70^{-/-} mast cell with a test agent, and determining the effect, if any, of the test agent on degranulation by the SWAP-70^{-/-} cell. Methods of determining mast cell degranulation are described above. The invention further provides agents identified by these methods, and compositions, including pharmaceutical compositions, comprising the agent.

Immature homozygous SWAP-70 knockout mast cells of the invention are useful to further test agents identified by a subject screening method to determine whether an agent identified by a subject method inhibits the residual capability of the immature SWAP-70 homozygous knockout mast cell to degranulate. Such agents are of interest to inhibit or reduce mast cell degranulation.

Agents

The present invention further provides active agents that modulate an activity or level of SWAP-70 in a mast cell or basophil. In some embodiments, an agent is one that is identified by an assay of the invention, as described above. Agents are useful to inhibit or reduce mast cell degranulation.

In many embodiments, the agent is a small molecule, e.g., a small organic or inorganic compound having a molecular weight of more than 50 and less than about 2,500 daltons. Agents may comprise functional groups necessary for structural interaction with proteins, particularly hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Agents are also found among biomolecules including peptides, saccharides, fatty acids, steroids, purines, pyrimidines, derivatives, structural analogs or combinations thereof.

In some embodiments, an active agent is a peptide. Suitable peptides include peptides of from about 3 amino acids to about 50, from about 5 to about 30, or from about 10 to about 25 amino acids in length. In some embodiments, a peptide exhibits one or more of the following activities: inhibits binding of SWAP-70 to a SWAP-70 interacting protein; inhibits SWAP-70 binding to a second SWAP-70 molecule; inhibits phosphorylation of SWAP-70 by a kinase, e.g., a SYK kinase; inhibits an enzymatic activity of SWAP-70 ; inhibits a signal transduction activity of SWAP-70. In some embodiments, a peptide has a sequence of from about 3 amino acids to about 50, from about 5 to about 30, or from about 10 to about 25 amino acids of a naturally-occurring SWAP-70 protein.

Peptides can include naturally-occurring and non-naturally occurring amino acids. Peptides may comprise D-amino acids, a combination of D- and L-amino acids, and various “designer” amino acids (e.g., β-methyl amino acids, Cα-methyl amino acids, and Nα-methyl amino acids, etc.) to convey special properties to peptides. Additionally, peptide may be a cyclic peptide. Peptides may include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics may be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analog; amide bond isostere; tretrazol; and the like.

A peptide may be a depsipeptide, which may be a linear or a cyclic depsipeptide. Kuisle et al. (1999) Tet. Letters 40:1203-1206. “Depsipeptides” are compounds containing a sequence of at least two alpha-amino acids and at least one alpha-hydroxy carboxylic acid, which are bound through at least one normal peptide link and ester links, derived from the hydroxy carboxylic acids, where “linear depsipeptides” may comprise rings formed through S—S bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. “Cyclic depsipeptides” are peptides containing at least one ring formed only through peptide or ester links, derived from hydroxy carboxylic acids.

Peptides may be cyclic or bicyclic. For example, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art

The term “bicyclic” refers to a peptide in which there exists two ring closures. The ring closures are formed by covalent linkages between amino acids in the peptide. A covalent linkage between two nonadjacent amino acids constitutes a ring closure, as does a second covalent linkage between a pair of adjacent amino acids which are already linked by a covalent peptide linkage. The covalent linkages forming the ring closures may be amide linkages, i.e., the linkage formed between a free amino on one amino acid and a free carboxyl of a second amino acid, or linkages formed between the side chains or “R” groups of amino acids in the peptides. Thus, bicyclic peptides may be “true” bicyclic peptides, i.e., peptides cyclized by the formation of a peptide bond between the N-terminus and the C-terminus of the peptide, or they may be “depsi-bicyclic” peptides, i.e., peptides in which the terminal amino acids are covalently linked through their side chain moieties.

A desamino or descarboxy residue can be incorporated at the terminii of the peptide, so that there is no terminal amino or carboxyl group, to decrease susceptibility to proteases or to restrict the conformation of the peptide. C-terminal functional groups include amide, amide lower alkyl, amide di(lower alkyl), lower alkoxy, hydroxy, and carboxy, and the lower ester derivatives thereof, and the pharmaceutically acceptable salts thereof.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, S., Bioconjugate Chem., 6:150-165 (1995); Monfardini, C, et al., Bioconjugate Chem., 6:62-69 (1995); U.S. Pat. Nos. 4,640,835; 4,496,689; 4,301,144; 4,670,417; 4,791,192; 4,179,337 or WO 95/34326.

Another suitable agent for reducing an activity of SWAP-70 is a peptide aptamer. Peptide aptamers are peptides or small polypeptides that act as dominant inhibitors of protein function. Peptide aptamers specifically bind to target proteins, blocking their function ability. Kolonin and Finley, PNAS (1998) 95:14266-14271. Due to the highly selective nature of peptide aptamers, they may be used not only to target a specific protein, but also to target specific functions of a given protein (e.g. a signaling function). Further, peptide aptamers may be expressed in a controlled fashion by use of promoters which regulate expression in a temporal, spatial or inducible manner. Peptide aptamers act dominantly; therefore, they can be used to analyze proteins for which loss-of-function mutants are not available.

Peptide aptamers that bind with high affinity and specificity to a target protein may be isolated by a variety of techniques known in the art. Peptide aptamers can be isolated from random peptide libraries by yeast two-hybrid screens (Xu et al., PNAS (1997) 94:12473-12478). They can also be isolated from phage libraries (Hoogenboom et al., Immunotechnology (1998) 4:1-20) or chemically generated peptides/libraries.

Intracellularly expressed antibodies, or intrabodies, are single-chain antibody molecules designed to specifically bind and inactivate target molecules inside cells. Intrabodies have been used in cell assays and in whole organisms. Chen et al., Hum. Gen. Ther. (1994) 5:595-601; Hassanzadeh et al., Febs Lett. (1998) 16(1, 2):75-80 and 81-86. Inducible expression vectors can be constructed with intrabodies that react specifically with SWAP-70 protein. These vectors can be introduced into model organisms and studied in the same manner as described above for aptamers.

In some of the invention, the active agent is an agent that modulates, and generally decreases or down regulates, the expression of the gene encoding SWAP-70 in the host. Such agents include, but are not limited to, antisense RNA, interfering RNA, ribozymes, and the like.

In some embodiments, the active agent is an interfering RNA (RNAi). RNAi includes double-stranded RNA interference (dsRNAi). Use of RNAi to reduce a level of a particular mRNA and/or protein is based on the interfering properties of double-stranded RNA derived from the coding regions of gene. In one example of this method, complementary sense and antisense RNAs derived from a substantial portion of the SWAP-70 gene are synthesized in vitro. The resulting sense and antisense RNAs are annealed in an injection buffer, and the double-stranded RNA injected or otherwise introduced into the subject (such as in their food or by soaking in the buffer containing the RNA). See, e.g., WO99/32619. In another embodiment, dsRNA derived from a SWAP-70 gene is generated in vivo by simultaneous expression of both sense and antisense RNA from appropriately positioned promoters operably linked to SWAP-70 coding sequences in both sense and antisense orientations.

Antisense molecules can be used to down-regulate expression of the gene encoding SWAP-70 in cells. Antisense compounds include ribozymes, external guide sequence (EGS) oligonucleotides (oligozymes), and other short catalytic RNAs or catalytic oligonucleotides which hybridize to the target nucleic acid and modulate its expression.

The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al, (1993), supra, and Milligan et al, supra.) Preferred oligonucleotides are chemically modified from the native phosphodiester structure, in order to increase their intracellular stability and binding affinity. A number of such modifications have been described in the literature, which modifications alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The β-anomer of deoxyribose may be used, where the base is inverted with respect to the natural α-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Exemplary modified oligonucleotide backbones that do not include a phosphorus atom therein have backbones that are formed by short chain alkyl or cycloalkyl internucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Oligonucleotides having a morpholino backbone structure (Summerton, J. E. and Weller D. D., U.S. Pat. No. 5,034,506) or a peptide nucleic acid (PNA) backbone (P. E. Nielson, M. Egholm, R. H. Berg, O. Buchardt, Science 1991, 254: 1497) can also be used. Morpholino antisense oligonucleotides are amply described in the literature. See, e.g., Partridge et al. (1996) Antisense Nucl. Acid Drug Dev. 6:169-175; and Summerton (1999) Biochem. Biophys. Acta 1489:141-158.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

Formulations, Dosages, and Routes of Administration

The invention provides formulations, including pharmaceutical formulations, comprising an agent that reduces a level and/or an activity of SWAP-70. In general, a formulation comprises an effective amount of an agent that reduces a level and/or an activity of SWAP-70. An “effective amount” means a dosage sufficient to produce a desired result, e.g., a reduction in a level and/or an activity of SWAP-70, a reduction in mast cell degranulation, a reduction in histamine levels. Generally, the desired result is at least a reduction a level and/or an activity of SWAP-70 as compared to a control.

Formulations

In the subject methods, the active agent(s) may be administered to the host using any convenient means capable of resulting in the desired reduction in a level and/or an activity of SWAP-70. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Other modes of administration will also find use with the subject invention. For instance, an agent of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington3 s Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Dosages

Although the dosage used will vary depending on the clinical goals to be achieved, a suitable dosage range is one which provides up to about 1 μg to about 1,000 μg or about 10,000 μg of an agent that reduces a level and/or an activity of SWAP-70 can be administered in a single dose. Alternatively, a target dosage of an agent that reduces a level and/or an activity of SWAP-70 can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of Administration

An agent that reduces a level and/or an activity of SWAP-70 is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intratumoral, subcutaneous, intradermal, topical application, intravenous, rectal, nasal, oral and other parenteral routes of administration. Routes of administration may be combined, if desired, or adjusted depending upon the agent and/or the desired effect. The composition can be administered in a single dose or in multiple doses.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrastemal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as an allergic hypersensitivity. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits with unit doses of the active agent, e.g. in oral or injectable doses, are provided. In such kits, in addition to the containers containing the unit doses will be an informational package insert describing the use and attendant benefits of the drugs in treating pathological condition of interest. Preferred compounds and unit doses are those described herein above.

Combination Therapies

The present invention also provides methods of treating an individual having a disorder associated with mast cell degranulation, comprising administering an agent of the invention and a second therapeutic agent. Therapeutic agents include, but are not limited to, an anti-histamine, an anti-inflammatory agent, a leukotriene synthesis inhibitor, an immunosuppressant, a bronchodilator, a vasoconstrictor, a decongestant, a leukotriene inhibitor, and the like.

Suitable therapeutic agents which can be used in combination therapies with an agent of the instant invention include, but are not limited to, antihistamines such as loratadine (Claritin®), fexofenadine (Allegra®), terfenadine; astemizole, cetirizine, hydroxyzine, diphenhydramine; leukotriene synthesis inhibitors zileutron (Zyflo®); leukotriene receptor antagonists such as zafirlukast (Accolate®), and montelukast; β-adrenergic agonists such as epinephrine, isoproterenol, isoetharine, metaproterenol, albuterol, terbutaline, bitolterol, pirbuterol, and salmeterol; proinflammatory cytokine antagonists; proinflammatory cytokine receptor antagonists; anti-CD23; anti-IgE; anticholinergics such as atropine and ipratropium bromide; immunomodulating drugs; glucocorticosteroids; steroid chemical derivatives; anti-cyclooxygenase agents; anti-cholinergic agents; methylxanthines, cromones; anti-CD4 reagents; anti-IL-5 reagents; anti-thromboxane reagents; anti-serotonin reagents; ketotiphen; cytoxin; cyclosporin; methotrexate; macrolide antibiotics; heparin; and low molecular weight heparin.

An agent that reduces a level or an activity of a SWAP-70 protein can also be administered in combination therapy with an agent that inhibits degranulation of a subject SWAP-70^{-/-} cell, as described above.

Methods of Reducing Degranulation

The present invention provides methods of reducing degranulation in a degranulation competent cell, e.g., a mast cell. The methods generally involve reducing a level and/or an activity of a SWAP-70 protein in the cell.

The methods involve administering to an individual having a disorder associated with mast cell degranulation, or caused by mast cell degranulation, or a disorder of which mast cell degranulation is a symptom, an effective amount of an agent that reduces a level and/or an activity of SWAP-70.

The efficacy of the treatment can be monitored according to clinical protocols well known in the art for monitoring the treatment of allergic disorders. For example, such clinical parameters as allergy symptoms (itching, sneezing, coughing, respiratory congestion, rhinorrhea, skin eruption, etc.), assays and skin prick tests (wheal and flare response) to known allergens and serum levels of IgE and allergy-associated cytokines (e.g., interleukin-4, interleukin-5) can be monitored for determining efficacy. Indicators of efficacy of the treatment can include a reduction in severity and/or absence of symptoms, an increase in the number of symptom-free days per time period (e.g., per month) and/or a reduction in the need for conventional medications such as decongestants, anti-histamines, mast cell stabilizers and corticosteroids.

If the treatment of this invention is carried out in conjunction with immunotherapy, efficacy can be evaluated by observing an increase in tolerated dose of a given allergen(s). These parameters can be monitored weekly or monthly, as well as at greater time intervals (e.g., every 3-6 months). In a particular example, clinical parameters that can be monitored for asthma can include the number and severity of attacks as determined by symptoms of wheezing, shortness of breath and coughing. The measurement of airway resistance by the use of respiratory spirometry, the extent of disability and the dependence on immunosuppressive medications or bronchodilators can also be determined.

The efficacy of treatment for preventing an allergic disorder in a subject not known to have an allergic disorder, but known to be at risk of developing an allergic disorder, can be determined by evaluating clinical parameters such as allergy symptoms (itching, sneezing, coughing, respiratory congestion, rhinorrhea, skin eruption, etc.), assays and skin prick tests (wheal and flare response) to known allergens and serum levels of IgE and allergy-associated cytokines (e.g., interleukin-4, interleukin-5), over time following administration of the nucleic acid or fusion protein of this invention. This time interval can be very short (i.e, minutes/hours) or very long (i.e., years/decades). The determination of who would be at risk for the development of an allergic disorder would be made based on current knowledge of the known risk factors for a particular allergic disorder as would be familiar to clinicians and researchers in this field, such as a particularly strong family history of an allergic disorder or exposure to or acquisition of factors or conditions (i.e., environmental) which are likely to lead to development of an allergic disorder.

Disorders Amenable to Treatment

Disorders amenable to treatment using the subject methods and agents include any allergic disorder that involves mast cell degranulation. The term “allergic disorder” generally refers to a disease state or syndrome whereby the body produces an immune response to environmental antigens comprising immunoglobulin E (IgE) antibodies which evoke allergic symptoms such as itching, sneezing, coughing, respiratory congestion, rhinorrhea, skin eruptions and the like, as well as severe reactions, such as asthma attacks and systemic anaphylaxis. Examples of allergic diseases and disorders which can be treated or prevented by the methods of this invention include, but are not limited to, drug hypersensitivity, allergic rhinitis, bronchial asthma, ragweed pollen hayfever, anaphylactic syndrome, urticaria, angioedema, atopic dermatitis, erythema nodosum, erythema multiforme, Stevens-Johnson Syndrome, cutaneous necrotizing venulitis, bullous skin diseases, allergy to food substances and insect venom-induced allergic reactions, as well as any other allergic disease or disorder.

Subjects Amenable to Treatment

Subjects amenable to treatment using the methods and agents described herein include individuals who are known to have allergic hypersensitivity to one or more allergens. Subjects amenable to treatment include those who have any of the above-mentioned allergic disorders. Also amenable to treatment are subjects that are at risk of having an allergic reaction to one or more allergens.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Involvement of SWAP-70 in Mast Cell Degranulation

Materials and Methods

Cell Culture and other Methods

BMC and BMMC cultures were established from 8-weeks old mice from 4 drops blood or bone marrow cell suspension after purifying cells by ficoll gradients (Refs. 5-8). The initial cultures, established in 24-well plates, of BMC contained about five-fold less mast cells than the BMMC cultures, but proliferation was at the same rate. Cells were continuously passaged for at least 5 weeks in IMDM, supplemented with 10% fetal calf serum and 50 μg/ml IL-3, until the cultures were homogenous in appearance. Mast cells were examined by staining with toluidine blue (weak) and berberine sulfate (no staining), and for surface expression of c-kit and FcεRI/IgE complexes by FACS using anti-c-kit-FITC or anti-IgE-FITC antibodies. These cultures were kept alive for at least several months.

Analysis of peritoneal fluid by Percol gradient centrifugation used step-gradients (increments 0.005) with densities of Percol in PBS, 2% FCS from 1.07 to 1.09. Cells accumulating in the pellet and those banding at 1.09 and 1.085 densities stained intensively with toluidine blue and with berberine sulfate and are regarded as mature mast cells. Assays for apoptosis used staining of cells with propidium iodide or anti annexin V antibodies as described (Ref. 15). Immunofluorescence and Western blotting were performed as described (Ref. 24).

Berberin Sulfate and Toluidine Blue Staining

For staining with berberin sulfate, cytospins of mast cells were fixed (15 min in 3:1 ethanol: acetic acid), washed for 15 min in 100% ethanol, and allowed to air-dry. After a 10 min wash in distilled water, slides were incubated 20 min with 0.02% berberin sulfate in 1% citric acid, pH 4. Slides were washed in 1% citric acid, air-dried, fluoromount added, and analysed under the fluorescence microscope. For toluidine blue staining, cytospins were fixed with 50% ethanol for 15 min, and incubated for 30 min with 0.1% toluidine blue (in 30% ethanol). Slides were washed with water, allowed to dry, and cells investigated by light microscopy.

Exocytosis Assays

1×10⁵ cells/sample were incubated on ice for 45 min in medium containing 10 μg/ml mouse IgE. After centrifugation and one wash with PBS, 2% fetal calf serum, cells were resuspended in degranulation buffer (119 mM NaCl, 5 mM KCl, 0.4 mM MgSO₄, 25 mM PIPES pH 7.2, 5.6 mM glucose, 1 mM CaCl₂, 0.1% ultrapure BSA), and anti-IgE added (2 μg/ml), washed twice and resuspended in this buffer. After incubation for 15 min, cells were pelleted, and, for hexosaminidase measurements, 25 μl supernatant incubated with 25 μl 1 mM mM 4-methylumbelliferyl-N-acetyl-β-D-glucosaminide, dissolved in 0.2 M citrate buffer pH 4.5, for 2 hours at 37° C. The reaction was stopped by addition of 150 μl 1 M Tris (base), and fluorescence (360-450 nm) measured in a microtiter device. In controls, either IgE or anti-IgE, or both, were omitted, or 2 μM ionomycin used instead of anti-IgE. To measure the total hexosaminidase content, triton lysis (0.4%) of the cells was performed, and the supernatant assayed.

In the serotonin release assay, mast cells were cultivated for 16 hours with 1 μCi/ml ³H-hydroxytryptamine binoxalate 5-[1,2-3H(N)]₇, and washed twice with 1×10⁵ cells were used in the IgE/anti-IgE treatment protocol as described for the hexosaminidase release assay. The ³H radioactivity present in 50 μl supernatant was measured in a scintillation counter. Stimulation by antigen, i.e. crosslinking of anti-DNP IgE by DNP-BSA was used in the following experiments. Release of IL-6, TNF-α, and LTC4 was measured by ELISA in cell supernatants. Cytokines released from the cells were measured during a period of 30 min to 4 h after stimulation, in order to only measure newly synthesized cytokines. The annexin-V binding assay was done as described (Ref. 22). All experiments were performed at least in triplicates.

Mast Cells Express SWAP-70

In nuclear extracts from a wide variety of cells and tissues, SWAP-70 was found only in those from activated B cells (Refs. 11,12). In tissue sections including brain, kidney, skin, prostate, small intestine, bone marrow, thymus, and spleen, however, a few cells were observed to stain outside of the B lymphocyte compartment. To test whether these cells include mast cells, we established cultures of mast cells (Refs. 5,7,8). For blood-derived mast cells (BMC) or bone marrow-derived mast cells (BMMC) cells we collected cells from the respective organs and cultivated them in medium containing IL-3. The cells were passaged every 5 days, and after 5 weeks the cultures had a homogenous appearance.

The cells were analysed by FACS for binding stem cell factor (SCF) and IgE, and more than 98% of the cells were positive for both, showing the same fluorescence intensity. This pattern did not change upon prolonged cultivation, and did not differ between BMC and BMMC. The cells stained weakly with toluidine blue, and did not stain with berberine sulfate. On electron micrographs the cells showed granules and a monolobal nucleus typical for mast cells (Refs. 2,17). The granules, however, are not yet of high electron density, indicative of an immature stage of the cells.

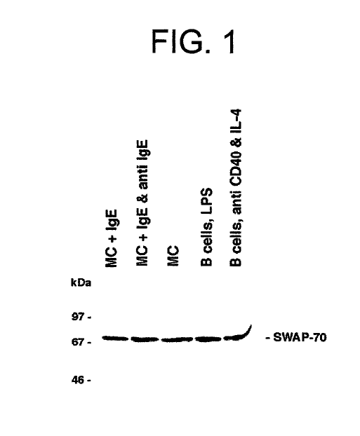

Total cell lysates from BMC or BMMC cultures were analyzed for the presence of SWAP-70 protein by immunoblotting with affinity-purified anti-SWAP-70 antibodies (Refs. 11,12), as shown in FIG. 1. In control lanes, the identical amount of protein from total cell lysates of splenic B cells, activated by either lipopolysaccharide or anti-CD-40 antibody plus IL-4 (Refs. 11,12), were loaded. Both, proliferating BMC and BMMC expressed about as much SWAP-70 as activated B cells. Thus, at least some of the rare non-B cells that express SWAP-70 are mast cells.

Incubation of the mast cells with either IgE or with IgE and anti-IgE for activation did not alter the level of SWAP-70 protein. In contrast to B cells, in proliferating mast cells of either BMC or BMMC type, SWAP-70 was exclusively localized in the cytoplasm, as determined by immunofluorescence. Nuclear localization of SWAP-70 was also neither observed after yirradiation, UV-irradiation, or activation of mast cells with an IgE antibody and crosslinking with anti-IgE antibody to trigger degranulation. In mast cells, SWAP-70 may either be excluded from the nucleus, e.g. by masking or otherwise inactivating the three nuclear localization signals present in SWAP-70. Or the balance between nuclear import and export may be dramatically shifted in favor of export. At any rate, SWAP-70 intracellular localization is cell type-specific. In most mast cells that were γ-irradiated or activated by IgE/anti-IgE, more intense staining was observed at the cytoplasmic membrane, indicative of membrane-association of SWAP-70. As these are very different treatments, it indicates that the putative membrane association of SWAP-70 in mast cells may occur in different signaling pathways.

Mature Mast Cells in SWAP-70 Deficient Mice Degranulate Normally

To study the role of SWAP-70 in mast cell degranulation, and in mast cells in general, we used recently created mice deficient in the protein (Ref. 15). In these mice, the first exon and part of the 5′ UTR were removed in both alleles by gene targeting. The homozygous SWAP-70^{ko/ko} mice express SWAP-70 neither in activated splenic B cells nor in mast cells (Ref. 15).

Mast cells derived from the SWAP-70^{ko/ko} mice looked very similar to wild type mast cells in electron microscopy, and the flow cytometry profiles of the FcεRI receptor, and staining characteristics with toluidine blue and berberine sulfate were the same for the SWAP-70 deficient and wild type cells. Peritoneal fluid was analyzed from each 6 wild type or SWAP-70^{ko/ko} mice by Percoll gradient centrifugation (1.07-1.09 density) and toluidine blue staining of cells in the pellet (high density mature mast cells) and at the 1.09 and 1.085 densities (mature mast cells of lower densities). SWAP-70^{ko/ko} mice contained 8-fold less high density mast cells, and 2-fold less of the mature mast cells of lower density.

We also counted in peritoneal fluid from each of six 6-weeks old wild type and SWAP-70^{ko/ko} mice the total numbers of mast cells by toluidine blue staining, and found that SWAP-deficient mice had 1.9×less mast cells (wild type: 864 of 7518 total cells counted (11.5%); SWAP-70^{ko/ko}: 442 of 7412 (5.9%)).

As young mice do not develop significant numbers of mast cells that bear high-density granules, these numbers ought to represent mature mast cells with low-density granules. This is also indicated by weaker than wild type staining of SWAP-70^{ko/ko} peritoneal mast cells with berberin sulfate, a dye that brightly stains high-density mature mast cells. In addition, we counted mast cells in the skin of the ear, an organ particularly rich in mast cells, of 5-weeks old mice. Consistent with our other observations, the SWAP-70^{ko/ko} samples contained reduced numbers of toluidine blue-stained mast cells (wild type: 117 mast cells per mm²; 584 of 1632 total cells counted (35.8%); SWAP-70^{ko/ko}: 53 mast cells per mm²; 266 of 1326 (20.0%)).

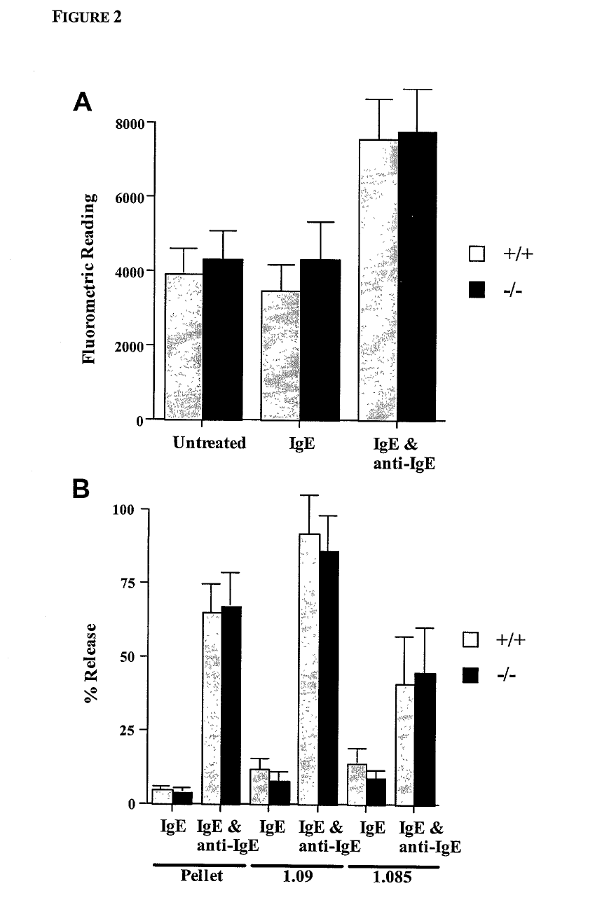

The difference in numbers notwithstanding, mature mast cells from SWAP-70^{ko/ko} mice degranulated as efficiently as wild type mast cells. Degranulation was triggered by anti-IgE mediated crosslinking of IgE bound to the FcεRI, and measured in the hexosaminidase assay, as shown in FIG. 2A. This result was confirmed by separating mature mast cells from other peritoneal cells into three populations, as shown in FIG. 2B. Peritoneal cells from five 6-months old mice of each genotype, wild type or SWAP-70^{ko/ko}, were pooled and subjected to Percoll gradient centrifugation. Cells were collected from the pellet and at the 1.09 and 1.85 densities, washed, and counted. The three gradient fractions contained almost exclusively mast cells as judged by staining with toluidine blue. Their degranulation capacity was not dependent on the presence of SWAP-70, as shown in FIG. 2B. Similar results were obtained with peritoneal cells from younger mice (6 or 8 weeks old).

Together, these results demonstrate that SWAP-70^{ko/ko} mice develop mature mast cells at numbers lower than wild type mice, but that those mast cells which reach the mature stage respond to FcεRI crosslinking by degranulation.

SWAP-70 Deficient Mast Cells Respond Normally to IL-3 and SCF

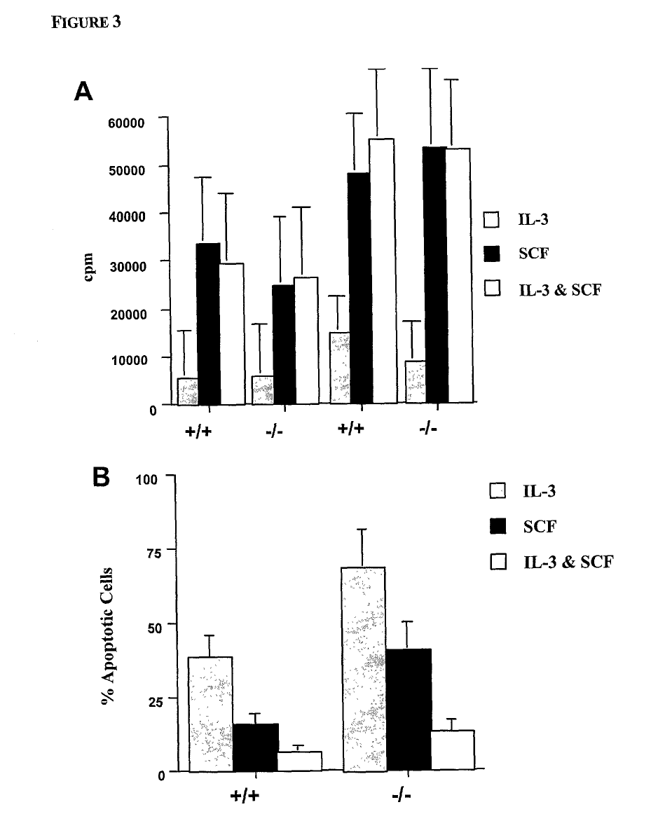

Because the number of mature mast cells was decreased it was likely that mast cells of earlier stages of development were affected by SWAP-70 deficiency. We thus analyzed BMC and BMMC cultures from the SWAP-70^{ko/ko} mice and wild type controls. Initially, we tested the effect on proliferation of IL-3 and SCF either alone or in combination, for both agents stimulate proliferation of mast cells. There was no difference in proliferation in either genotype, as shown in FIG. 3A. Since SCF also protects mast cells from apoptosis induced by γ-irradiation, we assayed the radiation sensitivity.

Mast cells from wild type and SWAP-70^{ko/ko} mice were cultured in the presence or absence of 100 ng/ml SCF, with or without IL-3, and irradiated with various doses (0-160 Gy). Subsequently, apoptosis was measured by staining with anti-annexin V and propidium iodine. SWAP-70^{ko/ko} cells were found to be slightly (2-fold) more sensitive to γ-irradiation than wild type cells, as shown in FIG. 3B. This elevated sensitivity was confirmed in a series of irradiation experiments in which mast cells were compared to other primary cell types, including thymocytes, preB cells, and fibroblasts. All of these non-mast cell were of identical sensitivity for γ-irradiation regardless of their SWAP-70 genotype. No increased sensitivity of SWAP-70 deficient mast cells for UV irradiation was observed. The SWAP-70 deficient cells were protected from 7-irradiation-induced apoptosis to a similar degree as wild type cells (3-fold by SCF; 5-fold by SCF and IL-3; as shown in FIG. 3B. This indicated that neither the SCF nor the IL-3 signaling pathways are affected by SWAP-70 deficiency.

Immature SWAP-70 Deficient Mast Cells are Impaired in Degranulation

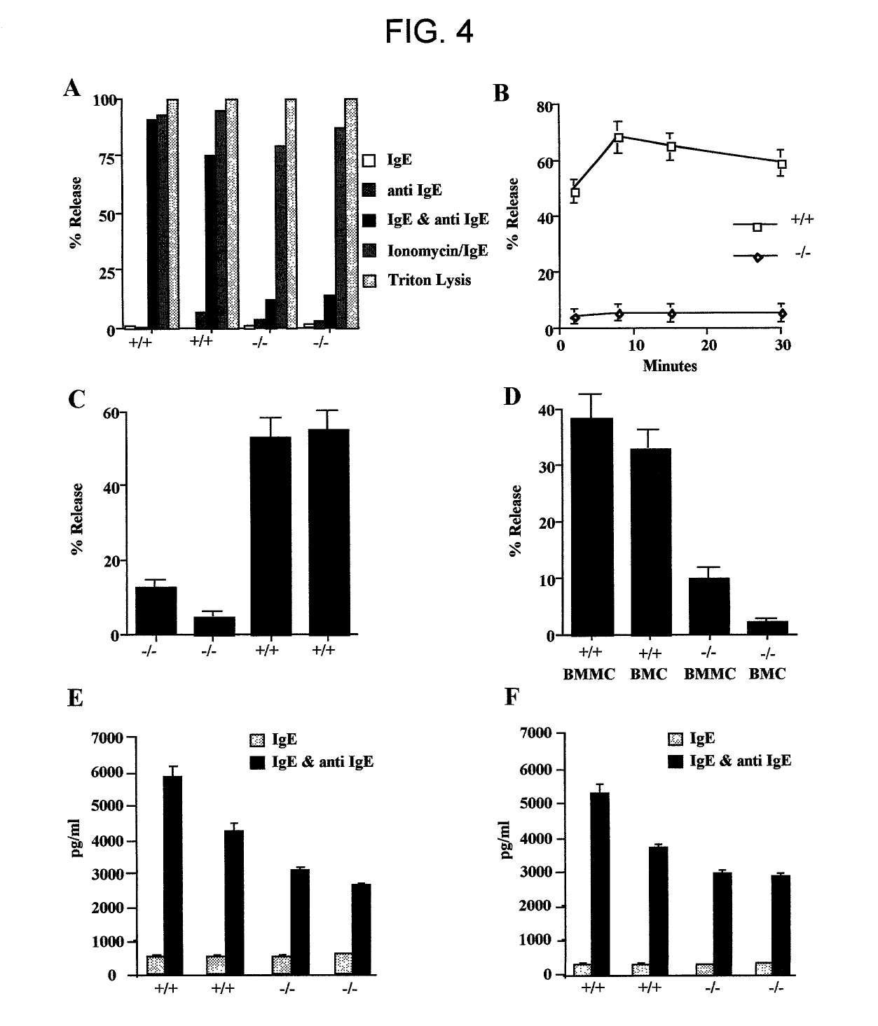

Although the BMC and BMMC cultures were identical in the phenotypes assessed, including FcεRI expression and formation of granules, their ability to degranulate was not. We conclude this from the degranulation experiments described below, the results of which are shown in FIGS. 4A-F. All degranulation experiments were performed with independent cultures from at least 4 mice of each genotype. We incubated the cells with a mouse IgE antibody, which is bound by the FcεRI and crosslinked with anti-IgE antibody. In negative controls, mast cells were incubated with either antibody alone, or buffer only.

To assess the capability of degranulation without the FcεRI signal mast cells were separately treated with ionomycin. Furthermore, lysis with the detergent Triton X-100 released the entire content of the mast cells and established the maximum value in the fluorometric read-out. Wild type mast cells did not degranulate when either IgE or anti-IgE was omitted, but they did when triggered by IgE/anti-IgE, or by ionomycin. In contrast, degranulation of the SWAP-70^{ko/ko} mast cells with IgE/anti-IgE was reduced to 15% of that of wild type mast cells. Triton-induced lysis gave nearly identical results for both genotypes, and ionomycin-triggered degranulation was very similar, indicating that the SWAP-70^{ko/ko} mast cells contain functional granules, which can release their content, as shown in FIG. 4A. Thus, not degranulation per se, but the IgE/FcεRI-signaling pathway leading to exocytosis, or the establishment of this pathway, requires SWAP-70. In FIG. 4B the degranulation reaction was stopped at various time points after addition of anti-IgE to the mast cells. At about 8 min wild type mast cells reached the maximum value, whereas SWAP-70^{ko/ko} mast cells gave only background, which remained flat even after 30 min. This indicates that degranulation is not just delayed but impaired in SWAP-70 deficient mast cells.

In the same assay, BMMC yielded similar results, as shown in FIG. 4C. In addition, we used other assays that measure mast cell degranulation. In one, the release of ³H-labeled serotonin is measured. In another, degranulation was triggered using an anti-DNP IgE antibody, and DNP-HSA for crosslinking. Then binding of annexin-V to the exocytosing granules was monitored by fluorescence activated cell sorting (FACS). Annexin-V binds in proportion to the extent of degranulation to granular membranes exposed in the process. Like for hexosaminidase, the release of serotonin and the binding of annexin-V were reduced to about 15-20% of wild type levels in SWAP-70^{ko/ko} mast cells, as shown in FIG. 4D. Release of other mast cell-derived mediators such as TNF-α, IL-6, and LTC4 was also consistently lower in SWAP-70^{ko/ko} than wild type mast cells, although not as pronounced as for hexosaminidase and serotonin, as shown in FIGS. 4E and F. This may reflect different levels of requirement of events downstream of IgE signaling for SWAP-70.

While all SWAP-70^{ko/ko} mast cells cultured with IL-3 were degranulation deficient for the initial 2-3 months of cultivation, after 5 months three out of four SWAP-70^{ko/ko} cultures degranulated at efficiencies closer to wild type (80%). Wild type cells maintained their initial degranulation capacity over this time period. Prolonged cultivation of primary cells in vitro may increase the chance of creating artifacts for example through selection processes occurring in cultures. One may speculate, however, that the recovery of the ability to degranulate may also reflect our observation in vivo, i.e., the accumulation—slow compared to wild type—of peritoneal mast cells with competence to degranulate.

One may further assume that mature peritoneal mast cells that are independent of SWAP-70 in their ability to degranulate, may even have ceased to express SWAP-70. Immunoblotting verified this assumption: by Percoll gradient centrifugation from total peritoneal cells we isolated from wild type mice high density mature mast cells that strongly stain with toluidine blue. These cells, regardless whether they were taken directly or at different time points after continuing treatment with anti IgE, do not express SWAP-70 at levels detectable by immunoblotting. Thus, in vivo the reemergence of degranulation capacity in mature, peritoneal SWAP-70^{ko/ko} mast cells developmentally coincides with the disappearance of SWAP-70 in the corresponding wild type mast cells.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of identifying an agent that reduces a level of a SWAP-70 polypeptide in a mast cell, the method comprising:
contacting a eukaryotic cell with a test agent; and
determining the effect, if any, of the test agent on the level of SWAP-70 polypeptide in the cell.

2. The method according to claim 1, wherein the agent reduces a level of SWAP-70 mRNA in the cell.

3. The method of claim 1, wherein the level of SWAP-70 polypeptide is determined using an antibody specific for SWAP-70.

4. A method of identifying an agent that reduces an activity of a SWAP-70 polypeptide in a mast cell, the method comprising:
contacting a eukaryotic cell with a test agent; and
determining the effect, if any, of the test agent on an activity of SWAP-70 in the cell.

5. The method of claim 4, wherein the activity of SWAP-70 is an interaction of SWAP-70 with a SWAP-70 interacting protein.

6. The method of claim 5, wherein the cell is a yeast cell, and wherein said determining is by a yeast two-hybrid method.

7. The method of claim 4, wherein the activity of SWAP-70 is an enzymatic activity.

8. The method of claim 4, wherein the cell is a mast cell, and the effect of the test agent on degranulation is determined.

9. A method of identifying an agent that reduces an activity of a SWAP-70 protein, the method comprising:
contacting a SWAP-70 polypeptide with a test agent; and
determining the effect, if any, of the test agent on an activity of the SWAP-70 polypeptide.

10. The method of claim 9, wherein the activity of SWAP-70 is an interaction of SWAP-70 with a SWAP-70 interacting protein other than SWAP-70.

11. The method of claim 9, wherein the activity of SWAP-70 is multimerization with at least a second SWAP-70 protein.

12. The method of claim 9, wherein the activity of SWAP-70 is an enzymatic activity.

13. A method of identifying an agent that inhibits phosphorylation of a SWAP-70 protein, the method comprising:
contacting a SWAP-70 polypeptide and a kinase that phosphorylates SWAP-70 with a test agent; and
determining the effect, if any, of the test agent on phosphorylation of the SWAP-70 polypeptide.

14. The method of claim 11, wherein said kinase is a SYK kinase.

15. A biologically active agent identified by a method according to any one of claims 1-14.

16. A pharmaceutical composition comprising a biologically active agent that reduces a level or an activity of a SWAP-70 protein; and a pharmaceutically acceptable excipient.

17. An isolated mast cell comprising a non-functional SWAP-70 allele.

18. The mast cell according to claim 17, wherein said mast cell is heterologous for said non-functional SWAP-70 allele.

19. The mast cell according to claim 17, wherein said mast cell is homologous for said non-functional SWAP-70 allele.

20. A method of inhibiting mast cell degranulation, comprising contacting a mast cell with an agent that reduces a level of or an activity of a SWAP-70 protein in the mast cell.

21. A method of treating a disorder associated with mast cell degranulation in an individual, the method comprising administering to the individual an agent that reduces a level or an activity of a SWAP-70 polypeptide in the cell.

22. The method according to claim 21, wherein the disorder is an allergic disorder.
